(19) United States
(12) Patent Application Publication
(10) Pub. No.: US 2025/0000754 A1
(43) Pub. Date: Jan. 2, 2025

(54) NEW FOAM FORMULATIONS FOR DERMATOLOGICAL APPLICATION, WITH PHASE REVERSAL

(71) Applicant: NSC PHARMA GMBH & CO.KG, Greven (DE)

(72) Inventor: Rolf DANIELS, Rottenburg (DE)

(21) Appl. No.: 18/274,143

(22) PCT Filed: Jan. 28, 2022
(86) PCT No.: PCT/EP2022/052006

§ 371 Date: May 3, 2024

(30) Foreign Application Priority Data
Jan. 28, 2021 (DE) DE102021000414.0

(51) Int. Cl. A61K 8/04; A61K 8/06; A61K 8/31; A61K 8/34; A61K 8/37; A61K 8/60; A61K 8/73; A61K 8/92

(52) U.S. Cl. CPC A61K 8/046; A61K 8/064; A61K 8/31; A61K 8/345; A61K 8/375; A61K 8/602; A61K 8/73; A61K 8/922

(57) ABSTRACT

The invention relates to novel W/O foam formulations, which are formed with phase reversal from aqueous O/W emulsions, and to their applications in cosmetics and dermatology.

### BACKGROUND OF THE INVENTION

The invention relates to novel W/O foam formulations, which are formed with phase reversal from aqueous O/W emulsions.

An essential feature of dermatological therapy is that the site of application and the site of action usually overlap. This circumstance brings the concrete galenics of dermal preparations to the foreground, since already the application of active ingredient-free bases has a considerable influence on the skin [R. Niedner and P. Ackermann, eds. Dermatika: Therapeutischer Einsatz, Pharmakologie und Pharmazie. Stuttgart: Wissenschaftliche Verlagsgesellschaft, 1992]. The spectrum of drugs for topical application ranges from liquid solutions and tinctures, over semi-solid ointments, creams and gels, to solid powders, with the semi-solid preparations being of greatest importance. A large number of semi-solid preparations are two-phase systems, which can be divided, on the basis of their phase position, into oil-in-water and water-in-oil emulsions, or O/W and W/O emulsions for short.

These emulsion types differ from one another in terms of their characteristic properties, which are largely determined by the respective external phase. For example, O/W emulsions are characterized by a light texture, which can be applied evenly, is absorbed quickly and thus has a particularly pleasant skin feel. However, due to their pronounced hydrophilic properties, such formulations have only a low substantivity and water resistance. Cosmetically, such formulations are used in daily moisturizing care, pharmaceutically they are of interest for the therapy of acute dermatoses or as vehicles for lipophilic active ingredients. In contrast, W/O emulsions have a sticky, greasy skin feel due to their lipophilic outer phase and are more difficult to apply, but they also have many advantageous properties. They form an occluding greasy film on the skin surface, which protects against external influences, is water-repellent and promotes active ingredient penetration [US 2017/0014315 A1]. Due to their pronounced substantivity and long-lasting skin care effect, W/O emulsions are ideally suited for the therapy of chronic dermatoses [1]. They are also widely used in waterproof sun protection products. So-called SWOP emulsions (SWitch Oil Phase) occupy an intermediate position here. During dermal application of these metastable O/W emulsions, a controlled phase inversion occurs from an O/W phase to a W/O phase [US 2017/0014315 A1]. This novel formulation concept promises to combine the excellent application properties of an O/W emulsion with the skin care benefits of a W/O emulsion [2]. SWOP emulsions are initially easy and uncomplicated to apply, yet leave a protective and water-resistant lipid film on the skin, which is accompanied by a long-lasting moisturizing effect and improved active ingredient penetration. This concept was developed for the manufacture of waterproof sun protection products that have a light skin feel and do not require any additional water-repellent ingredients. Improved consumer acceptance compared to conventional products has been demonstrated in a sensory test [EP 1917954 A1]. In addition, the pharmaceutical application of SWOP emulsions is conceivable. Especially in the therapy of large-area dermatoses and chronic skin diseases, the patient's compliance is largely dependent on the cosmetic properties of the dermal preparations to be applied. The use of suitable SWOP emulsions could significantly facilitate the application of the preparation while maintaining its efficacy.

Foaming creams represent another advantageous option for topical application [R. Daniels. “Dermopharmazie-Die richtige Galenik für kranke Haut”. In: Pharmazeutische Zeitung 154.24 (2009)). Due cosmetic to their properties, they are characterized by a high consumer acceptance. They can be applied almost contact-free and are therefore excellently suited for use on skin irritations and pain-sensitive wounds. The foam formulation also means that less product is generally applied, resulting in even distribution and rapid absorption. Due to their particularly light texture, they can also be easily distributed to hard-to-reach or hairy areas of the body [R. Daniels. “Basistherapeutika” In: Der Hautarzt 68.11 (2017), pp. 912-915.]. As aerosol foams, they are hygienic to dispense and protected from microbial contamination during use, which is why they usually do not require preservatives. This is also an advantage when used on reddened, irritated or even injured skin.

There is therefore a need for new formulations that combine the advantages of SWOP emulsions with those of stable foam formulations.

### SUMMARY OF THE INVENTION

The multiphase system of a SWOP emulsion according to the invention contains the following essential ingredients:
1. at least one physiologically compatible oil,
2. a W/O emulsifier,
3. an anionic co-surfactant,
4. a polymeric gelling agent,
5. water,
6. a propellant or propellant mixture.

### BRIEF DESCRIPTION OF THE DRAWINGS

FIG. 1 shows the change in this foam formulation over time.

### DETAILED DESCRIPTION OF THE INVENTION

Suitable W/O emulsifiers are, for example, sorbitan fatty acid esters or polyglycerol fatty acid esters, which can be used in concentrations of 2 to 8% (m/m). According to the invention, polyglyceryl-2-dipolyhydroxystearate (Dehymuls PGPH) is preferred. The W/O emulsifier is always a component of the lipophilic phase, which is mainly composed of a mixture of different lipids. These can be selected from the polar and non-polar oils or mixtures thereof. The lipid phase of the formulations according g to the invention is advantageously selected from the group of phospholipids and fatty acid triglycerides, from the group of propylene glycols or butylene glycols, fatty acid esters, from the group of natural waxes, of animal and vegetable origin, from the group of ester oils, from the group of dialkyl ethers and dialkyl carbonates, from the group of branched and unbranched hydrocarbons and waxes, and from the group of cyclic and linear silicone oils. Natural oils such as sunflower oil, apricot kernel oil, avocado oil, borage seed oil, safflower oil, peanut oil, hardened peanut oil, coconut oil, linseed oil, macadamia nut oil, almond oil, evening primrose oil, olive oil, palm oil, rapeseed oil, rice bran oil, castor oil, sea buckthorn oil, sesame oil, grape seed oil, shea butter, shorea butter and/or synthetic oils such as medium-chain triglycerides (e.g. caprylic/capric triglyceride, caprylic/capric/myristic/stearic triglycerides, C16-C18 triglycerides, propylene glycol dicaprylate/dicaprate, coco-glycerides as well as silicone oils (e.g., polydimethylsiloxanes, polymethylsiloxanes, polymethylphenyl-siloxanes, dimethiconols, alkyl dimethicones, alkyl methicones, cyclopentasiloxanes) can be used advantageously. Also particularly suitable are fatty alcohols. In addition, a viscosity-increasing addition by suitable long-chain fatty alcohols or various waxes is also possible. The preferred concentration of the oil mixture is 15 to 25% (m/m). Further auxiliaries such as colorants or complexing agents can also be added to the lipophilic phase.

The hydrophilic phase consists primarily of purified water. The co-surfactant (0.1 to 10% (m/m)) that may be present in the hydrophilic phase preferably belongs to the group of N-acyl amino acids, alkyl polyglycol ether citrates, or alkyl and alkenyl oligoglycosides. The most common example is Plantapon LGC Sorb, a mixture of lauryl glucoside and sodium lauryl glucose carboxylate. Other examples are the N-acyl amino acids sodium cocoyl glutamate (Plantapon ACGHC) and sodium stearoyl glutamate (Eumulgin SG).

The co-surfactant can be incorporated both initially into the lipophilic (li) and into the aqueous phase (aq). This could be demonstrated by means of various examples. No significant differences were found with regard to the optical and sensory properties, the change in electrical resistance, and the phase ratios before and after phase inversion.

This means that a suitable co-surfactant is mandatory for the preparation of a SWOP emulsion. It stabilizes the O/W phase position and prevents phase separation during phase inversion. The type of co-surfactant and the production method play a subordinate role. Consequently, other properties, such as the skin irritation potential of the co-surfactant or its compatibility with other ingredients, can also be taken into account.

Furthermore, the essential gelling agent is a component of the hydrophilic phase. Polyacrylates and polysaccharides are usually used in concentrations of 0.05 to 4% (m/m). In accordance with the invention, SWOP emulsions containing either sodium polyacrylate or xanthan gum are preferred.

In addition, a large number of other auxiliaries can be added to the water phase. So-called hydrotropes are water-soluble but comparatively polar organic compounds, which in turn influence the water solubility of other components. Under suitable circumstances, they can thus act as solubilizers. The SWOP emulsions of the invention contain glycerol, for example, but the use of ethanol or propylene glycol is also conceivable. The use of suitable preservatives is optional. Typical active ingredients used in cosmetic dermatology can easily be incorporated into the SWOP emulsions according to the invention. These include, for example, antioxidants, plant extracts, UV filters, urea, hyaluronic acid and selected provitamins and vitamins.

To produce a SWOP emulsion, the hydrophilic and lipophilic phases are first prepared separately and then combined and homogenized. Depending on the temperature, a distinction is made between three production methods: The so-called “cold/cold process” is carried out exclusively at room temperature (15 to 35° C.). In the “warm/cold process”, the lipophilic phase is heated to 70 to 85° C. during production, while in the “cold/warm process” the hydrophilic phase is heated accordingly. From an ecological point of view, the cold/cold process is to be preferred. Since the components of the formulation do not have to be heated or finally cold-stirred, energy and working time can be saved.

However, solid ingredients that can only be incorporated by heating may make it absolutely necessary to heat a phase.

For the production of foams containing active ingredients, the active ingredients can simply be added during manufacture. In the case of hydrophilic active ingredients, they are added to the aqueous phase; in the case of lipophilic active ingredients, they are added to the lipophilic phase.

By measuring a contact angle, phase inversion can be detected following application [EP 1917954 A1], [R. Kora′c et al., Colloids and Surfaces A: Physicochemical and Engineering Aspects 461 (2014), pp. 267-278].

For example, a small amount of SWOP emulsion is spread on a fingertip and a drop of water is placed on top. The contact angle of the water drop to the fingertip is then determined at regular intervals. The wetting angle is a measure of the interfacial tension and indirectly characterizes the phase position of an emulsion. A W/O emulsion leaves a lipophilic greasy film on the skin surface, resulting in poor wettability with water and a large angle. An O/W emulsion has a correspondingly smaller wetting angle.

The measured wetting angles after application of the SWOP emulsion increase rapidly and finally reach a constant final value above 60°. This abrupt change in the time course of the curve shows the phase inversion. Based on the inflection point, Amela Conesa et al. [EP 1917954 A1] determine the phase inversion point after 11.5 minutes, Kora′c et al. come to similar results. This phase inversion can be attributed to the evaporation of water, a tendency that is also known from conventional O/W emulsions. A direct comparison shows that the sudden increase in the wetting angle for a conventional O/W emulsion occurs only after a significantly longer period of time, of more than one hour [4]. The tendency to phase inversion can therefore not only be observed with SWOP emulsions, but they rightly occupy a special position due to the rapid speed of their phase inversion.

After the application of a SWOP emulsion, the transepidermal water loss is still lower after 24 hours than with an untreated skin surface. This corresponds to an improvement of the skin barrier function typical for W/O emulsions as well as an occlusive effect. In addition, SWOP emulsions have proven water resistance as measured by typical criteria for sunscreen products.

The rheological properties of SWOP emulsions differ in part considerably in their viscosity. Formulations with almost liquid and thus perceptible texture as well as highly viscous creamy variants are possible, with viscosities from 2,000 to 200,000 mPas. It can be seen that the viscosity increases in particular with increasing length of the carbon chain of the surfactant. This suggests that the co-surfactant within the formulation has not only an emulsifying function, but also a structure-forming and consistency-giving function. Kora′c et al. demonstrate a shear-thinning flow behavior with moderate thixotropy for SWOP emulsions. Compared to a conventional O/W emulsion, the two formulations differed mainly with respect to their yield point. That of the SWOP emulsion is significantly lower, suggesting that the gel structure is proportionally less pronounced. Under oscillating shear stress, the storage modulus G′ is always higher than the loss modulus G″, which is associated with a viscoelastic material behavior and underlines the gel character of the SWOP emulsions. Accordingly, these higher viscosity SWOP emulsions could also be regarded as creams.

In terms of sensory properties, the SWOP emulsion is similar to the O/W emulsion. However, the skin feel after application corresponds to that of a W/O emulsion due to the phase inversion. On microscopic examination, the SWOP emulsion is also comparable with an O/W emulsion, but has clear differences compared with a W/O emulsion.

SWOP emulsions are described generally as stable formulations. Over an observation period of three months, the examples investigated so far under isothermal storage conditions at room temperature (22±2° C.) show no quality-reducing instabilities. Even a so-called rocking test with interval-like temperature changes from −10 up to 40° C. or an increased load by centrifugation at 3,000 rpm does not lead to phase separation.

The polymeric gelling agent used is of central importance. In water, sodium polyacrylate is present in its deprotonated form. Due to the repulsion of the negatively charged carboxylate ions, the polymer swells and thus forms a gel structure.

Such carbomers react extremely sensitively to other electrolytes, these can reduce the stretching of the polymer by interacting with the carboxylate ions. In extreme cases, this can lead to a complete breakdown of the gel structure and a gel-sol transition. The anionic co-surfactant always present in SWOP emulsions thus leads to an already initially weakened gel structure with free bulk water. The interaction between sodium polyacrylate and the W/O emulsifier used is minimal. In addition, hydrophobic interactions between co-surfactant and emulsifier lead to the formation of mixed micelles. These are able to stabilize the oil droplets during production and storage. The final result is a stable equilibrium.

This can also be seen when looking at the SWOP emulsion under a microscope. The comparable O/W emulsion is characterized by very regular structures with well-formed droplets. In the SWOP emulsion, on the other hand, discontinuous layers of emulsifier and co-surfactant can be observed in the gelled water.

However, the drop-like structures are only incompletely enclosed here, which partly allows water to escape from the gel structure.

During application, this fragile equilibrium is disturbed by the electrolytes of the skin, the increased temperature of 32° C. and the force exerted during application itself. This causes the gel structure to collapse. Water released in the process evaporates, accelerated by the elevated temperature on the skin surface. This could result in a phase separation. However, a sufficiently high water content, emulsified by the contained W/O emulsifier, remains in the system so that a stable W/O emulsion can form. This reliable restabilization after phase inversion instead of phase separation is the decisive difference compared to conventional O/W emulsions.

The production of a SWOP emulsion is preferably carried out according to the cold/cold process described. With this manufacturing process, work can be carried out at room temperature throughout.

Since the components of the formulation do not have to be heated or finally cold-stirred, energy and working time can be saved with a qualitatively equivalent result. Various compositions according to the invention are disclosed in the examples described below.

In the cold/cold process, the lipophilic phase and the hydrophilic phase of the SWOP emulsion are initially produced separately and only joined together in the final step. For the lipophilic phase, all the lipids contained in the formulation are first weighed out and mixed homogeneously at, for example, 300 rpm with the aid of a stirrer. The W/O emulsifier is then added and stirred, also at 300 rpm, until a yellowish homogeneous liquid is obtained. For the hydrophilic phase, first the glycerol is given into a suitable preparation vessel and then the corresponding gel former is added. The gel former is then suspended homogeneously in the glycerol to obtain a lump-free gel later. If the formulation contains further cosolvents, a clear solution is first prepared separately with the required water. This aqueous solution or pure water is added in proportions to the suspension of the gel former so that a transparent, homogeneous gel 1 is formed. Alternatively, all water-soluble components can be mixed with the water and then the gel former dispersed in it without lumps and allowed to swell. The appropriate co-surfactant is then carefully added to prevent foaming. The lipophilic phase is added proportionally to the hydrophilic phase and incorporated with a suitable stirring tool to form a white, homogeneous emulsion. Any evaporated water can now be added before the emulsion is homogenized for three minutes using a homogenizer, e.g. Ultra Turrax at 8,000 rpm.

The emulsion is then filled into a tightly sealed container.

Before an emulsion prepared in this way can be examined and measured as described below, it must first rest for 24 hours to ensure comparable results.

### Foam Formulation

The production of foam formulations is realized with the help of appropriate aluminum aerosol cans.

Approximately 50 ml of the SWOP emulsion is filled into a previously tared aerosol can and sealed with a suitable valve plate with riser tube. The exact filling weight is then determined.

Based on this filling weight, the aerosol can is now charged with 3-10% (m/m), preferably approx. 5% (m/m), propellant gas in a conventional process. Finally, the foam formulation is shaken vigorously several times and provided with a suitable foam head.

Typically, n-butane with a saturation vapor pressure of 1.2 bar or propane-butane mixtures with higher saturation vapor pressures, e.g. 2.7 bar, are used as propellants. The examples show the exact compositions of the foam aerosols under consideration.

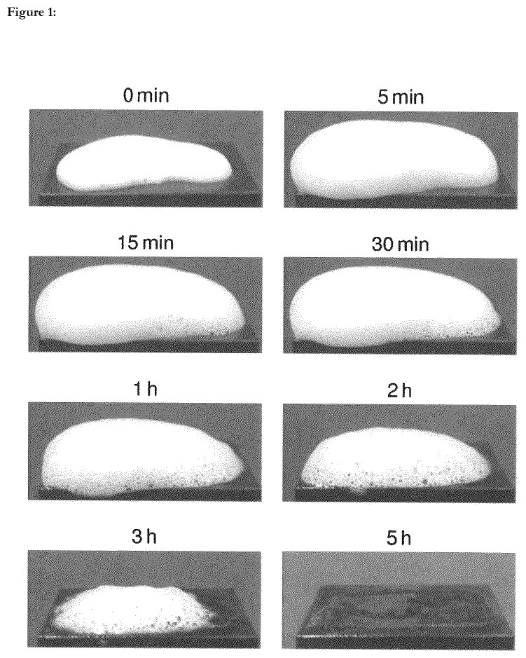

On subsequent removal from the aerosol can, a loose, white foam cream with a pleasantly light skin feel is obtained. FIG. 1 shows the change in this foam formulation over time. The foam volume continues to increase within the first few minutes and then remains virtually unchanged over a longer period (up to 5 hours), although the bubble size increases steadily.

After the foam collapses, a transparent, highly viscous liquid film remains.

The initial increase in volume makes it clear that foam formation only takes place during application due to the expansion of the propellant gas.

### Detection of Phase Inversion

The phase position of an emulsion is detected by determining the external phase. Various possibilities are described in the literature.

It has been found that the measurement of conductivity is an objective and quantitative way to determine the phase of an emulsion. It is based on the fact that aqueous systems, provided they contain small amounts of mobile charge carriers, are capable of conducting electric current, whereas oily systems have very low conductivity. Consequently, O/W emulsions, in contrast to W/O emulsions, exhibit a measurable conductivity of 10−7 to 10−4 s*cm⁻¹.

### EXAMPLES

The compositions according to the invention are further illustrated by the following examples, but are not limited thereto. These examples show the enormous range of components that can be used and their possible mixing ratios. Based on these examples, those skilled in the art can develop further compositions according to the invention without having to become inventive.

### Example 1: Basic Composition

| Substance | Amount |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 2: SWOP Emulsions with Variable Water Content

### Example 2a

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | |
| carboxylate + lauryl glucoside | |
| Glycerol | 2.5 g |
| Xanthan gum | 0.40 g |
| Highly purified water | 10.0 g |

### Example 2b

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Highly purified water | 20.0 g |

### Example 2c

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Highly purified water | 40.0 g |

### Example 2d

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 2e

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Highly purified water | 80.0 g |

### Example 2f

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Highly purified water | 100.0 g |

### Example 3: SWOP Emulsions with Variable Lipid Content

### Example 3a

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 3b

| | |
|---|---|
| Polyglyceryl-2-dipolyhydroxystearate | 2.0 g |
| Medium-chain triglycerides | 6.0 g |
| C12-15 alkyl benzoate | 6.0 g |
| Dicaprylylcarbonate | 4.0 g |
| Passion flower seed oil | 1.0 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 4: SWOP Emulsions with Lipids of Different Polarity

### Example 4a

| | |
|---|---|
| Polyglyceryl-2-dipolyhydroxystearate | 2.0 g |
| Castor oil | 8.0 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 4b

| | |
|---|---|
| Polyglyceryl-2-dipolyhydroxystearate | 2.0 g |
| C12-15 alkyl benzoate | 8.0 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 4c

| | |
|---|---|
| Polyglyceryl-2-dipolyhydroxystearate | 2.0 g |
| Coco-caprylate/caprate | 8.0 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 4d

| | |
|---|---|
| Polyglyceryl-2-dipolyhydroxystearate | 2.0 g |
| Dicaprylylether | 8.0 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 4e

| | |
|---|---|
| Polyglyceryl-2-dipolyhydroxystearate | 2.0 g |
| Light liquid paraffin | 8.0 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 5: SWOP Emulsions with Different Gelling Agents

### Example 5a

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.20 g |
| Highly purified water | 60.0 g |

### Example 5b

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 5c

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.80 g |
| Highly purified water | 60.0 g |

### Example 5d

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Highly purified water | 60.0 g |

### Example 5e

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Sodium polyacrylate | 0.40 g |
| Highly purified water | 60.0 g |

### Example 5f

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Sodium polyacrylate | 0.40 g |
| Sodium hydroxide sol. 8.1% | 0.40 g |
| Highly purified water | 60.0 g |

### Example 6: SWOP Emulsions with Variable W/O Emulsifier Content

### Example 6a

| | |
|---|---|
| Polyglyceryl-2-dipolyhydroxystearate | 1.0 g |
| Medium-chain triglycerides | 6.0 g |
| C12-15 alkyl benzoate | 6.0 g |
| Dicaprylylcarbonate | 4.0 g |
| Passion flower seed oil | 1.0 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 6b

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 6c

| | |
|---|---|
| Polyglyceryl-2-dipolyhydroxystearate | 4.0 g |
| Medium-chain triglycerides | 6.0 g |
| C12-15 alkyl benzoate | 6.0 g |
| Dicaprylylcarbonate | 4.0 g |
| Passion flower seed oil | 1.0 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 7: SWOP Emulsions with Different Co-Surfactants

### Example 7a

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 1.0 g |
| carboxylate + lauryl glucoside | |
| Glycerol | 2.5 g |
| Xanthan gum | 0.40 g |
| Highly purified water | 60.0 g |

### Example 7b

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 2.0 g |
| carboxylate + lauryl glucoside | |
| Glycerol | 2.5 g |
| Xanthan gum | 0.40 g |
| Highly purified water | 60.0 g |

### Example 7c

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium cocoyl glutamate | 1.0 g |
| Glycerol | 2.5 g |
| Xanthan gum | 0.40 g |
| Highly purified water | 60.0 g |

### Example 7d

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Disodium cetearyl succinate | 1.0 g |
| Glycerol | 2.5 g |
| Xanthan gum | 0.40 g |
| Highly purified water | 60.0 g |

### Example 7e

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium stearoyl glutamate | 1.0 g |
| Glycerol | 2.5 g |
| Xanthan gum | 0.40 g |
| Highly purified water | 60.0 g |

### Example 8: SWOP Emulsions with Variable Composition of the Emulsifier (W/O Emulsifier+Co-Surfactant)

### Example 8a

| | |
|---|---|
| Polyglyceryl-2-dipolyhydroxystearate | 3.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Glycerol | 2.5 g |
| Xanthan gum | 0.40 g |
| Highly purified water | 60.0 g |

### Example 8b

| | |
|---|---|
| Polyglyceryl-2-dipolyhydroxystearate | 2.5 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.5 g |
| carboxylate + lauryl glucoside | |
| Glycerol | 2.5 g |
| Xanthan gum | 0.40 g |
| Highly purified water | 60.0 g |

### Example 8c

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 1.0 g |
| carboxylate + lauryl glucoside | |
| Glycerol | 2.5 g |
| Xanthan gum | 0.40 g |
| Highly purified water | 60.0 g |

### Example 8d

| | |
|---|---|
| Polyglyceryl-2-dipolyhydroxystearate | 1.5 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 1.5 g |
| carboxylate + lauryl glucoside | |
| Glycerol | 2.5 g |
| Xanthan gum | 0.40 g |
| Highly purified water | 60.0 g |

### Example 8e

| | |
|---|---|
| Polyglyceryl-2-dipolyhydroxystearate | 1.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 2.0 g |
| carboxylate + lauryl glucoside | |
| Glycerol | 2.5 g |
| Xanthan gum | 0.40 g |
| Highly purified water | 60.0 g |

### Example 8f

| | |
|---|---|
| Polyglyceryl-2-dipolyhydroxystearate | 0.5 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 2.5 g |
| carboxylate + lauryl glucoside | |
| Glycerol | 2.5 g |
| Xanthan gum | 0.40 g |
| Highly purified water | 60.0 g |

### Example 8g

| | |
|---|---|
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 3.0 g |
| carboxylate + lauryl glucoside | |
| Glycerol | 2.5 g |
| Xanthan gum | 0.40 g |
| Highly purified water | 60.0 g |

### Example 9: SWOP Emulsions with Variable Glycerol Content

### Example 9a

| | |
|---|---|
| Glycerol | 0 g |
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Xanthan gum | 0.20 g |
| Highly purified water | 60.0 g |

### Example 9b

| | |
|---|---|
| Glycerol | 2.5 g |
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Highly purified water | 60.0 g |

### Example 9c

| | |
|---|---|
| Glycerol | 5.0 g |
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 5.0 g |
| Xanthan gum | 0.80 g |
| Highly purified water | 60.0 g |

### Example 10: SWOP Emulsions with Variable Alcohol Content

### Example 10a

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Ethanol | 10.0 g |
| Highly purified water | 50.0 g |

### Example 10b

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Ethanol | 20.0 g |
| Highly purified water | 40.0 g |

### Example 10c

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Ethanol | 30.0 g |
| Highly purified water | 30.0 g |

### Example 10d

| | |
|---|---|
| Polyglyceryl-2-dipolyhydroxystearate | 2.0 g |
| Medium-chain triglycerides | 3.0 g |
| C12-15 alkyl benzoate | 3.0 g |
| Dicaprylylcarbonate | 2.0 g |
| Passion flower seed oil | 0.5 g |
| Sodium lauryl glucose- | 0.75 g |
| carboxylate + lauryl glucoside | |
| Glycerol | 2.5 g |
| Xanthan gum | 0.40 g |
| Isopropanol | 10.0 g |
| Highly purified water | 50.0 g |

### Example 10e

| | | |
|---|---|---|
| Polyglyceryl-2-dipolyhydroxystearate | 2.0 | g |
| Medium-chain triglycerides | 3.0 | g |
| C12-15 alkyl benzoate | 3.0 | g |
| Dicaprylylcarbonate | 2.0 | g |
| Passion flower seed oil | 0.5 | g |
| Sodium lauryl glucose-carboxylate + lauryl | 0.75 | g |
| glucoside | | |
| Glycerol | 2.5 | g |
| Xanthan gum | 0.40 | g |
| Isopropanol | 20.0 | g |
| Highly purified water | 40.0 | g |

### Example 11: SWOP Emulsions with Variable Macrogol Content

### Example 11a

| | | |
|---|---|---|
| Polyglyceryl-2-dipolyhydroxystearate | 2.0 | g |
| Medium-chain triglycerides | 3.0 | g |
| C12-15 alkyl benzoate | 3.0 | g |
| Dicaprylylcarbonate | 2.0 | g |
| Passion flower seed oil | 0.5 | g |
| Sodium lauryl glucose-carboxylate + lauryl | 0.75 | g |
| glucoside | | |
| Glycerol | 2.5 | g |
| Xanthan gum | 0.40 | g |
| Polyethylene glycol 200 | 5.0 | g |
| Highly purified water | 55.0 | g |

### Example 11b

| | | |
|---|---|---|
| Polyglyceryl-2-dipolyhydroxystearate | 2.0 | g |
| Medium-chain triglycerides | 3.0 | g |
| C12-15 alkyl benzoate | 3.0 | g |
| Dicaprylylcarbonate | 2.0 | g |
| Passion flower seed oil | 0.5 | g |
| Sodium lauryl glucose-carboxylate + lauryl | 0.75 | g |
| glucoside | | |
| Glycerol | 2.5 | g |
| Xanthan gum | 0.40 | g |
| Polyethylene glycol 200 | 10.0 | g |
| Highly purified water | 50.0 | g |

### Example 11c

| | | |
|---|---|---|
| Polyglyceryl-2-dipolyhydroxystearate | 2.0 | g |
| Medium-chain triglycerides | 3.0 | g |
| C12-15 alkyl benzoate | 3.0 | g |
| Dicaprylylcarbonate | 2.0 | g |
| Passion flower seed oil | 0.5 | g |
| Sodium lauryl glucose-carboxylate + lauryl | 0.75 | g |
| glucoside | | |
| Glycerol | 2.5 | g |
| Xanthan gum | 0.40 | g |
| Polyethylene glycol 200 | 20.0 | g |
| Highly purified water | 40.0 | g |

### Example 11d

| | | |
|---|---|---|
| Polyglyceryl-2-dipolyhydroxystearate | 2.0 | g |
| Medium-chain triglycerides | 3.0 | g |
| C12-15 alkyl benzoate | 3.0 | g |
| Dicaprylylcarbonate | 2.0 | g |
| Passion flower seed oil | 0.5 | g |
| Sodium lauryl glucose-carboxylate + lauryl | 0.75 | g |
| glucoside | | |
| Glycerol | 2.5 | g |
| Xanthan gum | 0.40 | g |
| Polyethylene glycol 600 | 10.0 | g |
| Highly purified water | 50.0 | g |

### Example 12: SWOP Emulsions with Povidone

| | | |
|---|---|---|
| Polyglyceryl-2-dipolyhydroxystearate | 2.0 | g |
| Medium-chain triglycerides | 3.0 | g |
| C12-15 alkyl benzoate | 3.0 | g |
| Dicaprylylcarbonate | 2.0 | g |
| Passion flower seed oil | 0.5 | g |
| Sodium lauryl glucose-carboxylate + lauryl | | |
| glucoside | 0.75 | g |
| Glycerol | 2.5 | g |
| Xanthan gum | 0.40 | g |
| Polyvinylpyrrolidone 17 | 5.0 | g |
| Highly purified water | 55.0 | g |

### Example 13: SWOP Emulsions with Different Alkandiols

### Example 13a

| | | |
|---|---|---|
| Polyglyceryl-2-dipolyhydroxystearate | 2.0 | g |
| Medium-chain triglycerides | 3.0 | g |
| C12-15 alkyl benzoate | 3.0 | g |
| Dicaprylylcarbonate | 2.0 | g |
| Passion flower seed oil | 0.5 | g |
| Sodium lauryl glucose-carboxylate + lauryl | 0.75 | g |
| glucoside | | |
| Glycerol | 2.5 | g |
| Xanthan gum | 0.40 | g |
| 1,2-Propanediol | 10.0 | g |
| Highly purified water | 50.0 | g |

### Example 13b

| | | |
|---|---|---|
| Polyglyceryl-2-dipolyhydroxystearate | 2.0 | g |
| Medium-chain triglycerides | 3.0 | g |
| C12-15 alkyl benzoate | 3.0 | g |
| Dicaprylylcarbonate | 2.0 | g |
| Passion flower seed oil | 0.5 | g |
| Sodium lauryl glucose-carboxylate + lauryl | 0.75 | g |
| glucoside | | |
| Glycerol | 2.5 | g |
| Xanthan gum | 0.40 | g |
| 1,2-Propanediol | 30.0 | g |
| Highly purified water | 30.0 | g |

### Example 13c

| | | |
|---|---|---|
| Polyglyceryl-2-dipolyhydroxystearate | 2.0 | g |
| Medium-chain triglycerides | 3.0 | g |
| C12-15 alkyl benzoate | 3.0 | g |
| Dicaprylylcarbonate | 2.0 | g |
| Passion flower seed oil | 0.5 | g |
| Sodium lauryl glucose-carboxylate + lauryl | 0.75 | g |
| glucoside | | |
| Glycerol | 2.5 | g |
| Xanthan gum | 0.40 | g |
| 1,2-Hexanediol | 10.0 | g |
| Highly purified water | 50.0 | g |

### Example 14: SWOP Emulsion with Skin-Mimetic Lipids

2.0 g

| | | |
|---|---|---|
| Polyglyceryl-2-dipolyhydroxystearate | 2.0 | g |
| Medium-chain triglycerides | 5.0 | g |
| Isopropylmyristat | 5.0 | g |
| SLM* | 5.0 | g |
| Sodium lauryl glucose-carboxylate + lauryl | 0.8 | g |
| glucoside | | |
| Glycerol | 5.0 | g |
| Xanthan gum | 0.4 | g |
| Purified water ad | 100.0 | g |

*SLM {skin-mimetic lipids):Aqua, caprylic/capric triglycerides, hydrogenated phosphatidylcholine, pentylene glycol, glycerin, butyrospermum parkii (shea) butter, squalane, ceramide NP

### Example 15: SWOP-Emulsion with Urea

| Polyglyceryl-2-dipolyhydroxystearate | 2.0 | g |
|---|---|---|
| Dicaprylylether | 4.0 | g |
| Squalane | 2.0 | g |
| Sodium lauryl glucose-carboxylate + lauryl | 0.8 | g |
| glucoside | | |
| Urea | 15.0 | g |
| Glycerol | 5.0 | g |
| Xanthan gum | 0.4 | g |
| Lactic acid ad pH | 5.5 | |
| Purified water ad | 100.0 | g |

### Example 16: SWOP Emulsion with Skin-Mimetic Lipids and Urea

| Polyglyceryl-2-dipolyhydroxystearate | 2.0 | g |
|---|---|---|
| Dicaprylylether | 4.0 | g |
| Olive oil | 4.0 | g |
| SLM* | 2.0 | g |
| Sodium lauryl glucose-carboxylate + lauryl | 0.8 | g |
| glucoside | | |
| Urea | 5.0 | g |
| Glycerol | 5.0 | g |
| Xanthan gum | 0.4 | g |
| Lactic acid ad pH | 5.5 | |
| Purified water ad | 100.0 | g |

*SLM {skin-mimetic lipids): Aqua, caprylic/capric triglycerides, hydrogenated phosphatidylcholine, pentylene glycol, glycerin, butyrospermum parkii (shea) butter, squalane, ceramide NP

### Example 17: SWOP Emulsion with Dexpanthenol and Allantoin

| | | |
|---|---|---|
| Polyglyceryl-2-dipolyhydroxystearate | 2.0 | g |
| Medium-chain triglycerides | 5.0 | g |
| Isopropylmyristat | 5.0 | g |
| Sodium lauryl glucose-carboxylate + | 0.8 | g |
| lauryl glucoside | | |
| Allantoin | 0.2 | g |
| Dexpanthenol | 5.0 | g |
| Glycerol | 5.0 | g |
| Xanthan gum | 0.4 | g |
| Purified water ad | 100.0 | g |

### Example 18: SHOP Emulsion with Hyaluronic Acid and Vitamin E

| | | |
|---|---|---|
| Polyglyceryl-2-dipolyhydroxystearate | 2.0 | g |
| Olive oil | 3.0 | g |
| Octyldodecaol | 3.0 | g |
| Dicaprylylether | 4.0 | g |
| Sodium lauryl glucose-carboxylate + lauryl | 1.0 | g |
| glucoside | | |
| Hyaluronic acid | 0.2 | g |
| Tocopherol | 0.1 | g |
| 1,2-Pentanediol | 5.0 | g |
| Xanthan gum | 0.4 | g |
| Purified water ad | 100.0 | g |

### Example 19: SWOP Emulsion with Birch Bark Triterpenes

| | | |
|---|---|---|
| Polyglyceryl-2-dipolyhydroxystearate | 2.0 | g |
| Sunflower oil | 10.0 | g |
| Sodium lauryl glucose-carboxylate + lauryl | 0.8 | g |
| glucoside | | |
| Triterpene dispersion* | 10.0 | g |
| 1,2-Pentanediol | 5.0 | g |
| Xanthan gum | 0.4 | g |
| Purified water ad | 100.0 | g |

*Triterpene dispersion: Aqua, Helianthus annuus (sunflower) seed oil, hydrogenated phosphatidylcholine, Betula alba bark extract

### Example 20: Foam Formulations

The compositions according to the above examples 1-19 are transferred into suitable containers in a manner known in the art and a propellant serving as foaming agent is added. Particularly suitable have been found to be n-butane and propane/butane mixtures, which are preferably added to the compositions in 4-8% by weight. By actuating the foam head, which serves as a withdrawal valve, the respective foam is released and can be applied to the skin.

1. A composition for the preparation of a foamed W/O emulsion with phase inversion, comprising:
a) at least one physiologically compatible oil,
b) at least one W/O emulsifier,
c) at least one anionic co-surfactant,
d) at least one polymeric gelling agent,
e) water,
f) a propellant or propellant mixture.

2. The composition of claim 1, wherein natural oils such as sunflower oil and/or synthetic oils such as medium-chain triglycerides and/or silicone oils are present in a concentration from 15 to 25% (m/m) as physiologically compatible lipids.

3. The composition of claim 1, wherein fatty alcohols are present as physiologically compatible lipids.

4. The composition of claim 1, wherein the W/O emulsifier is sorbitan fatty acid ester and/or polyglycerol fatty acid ester, the W/O emulsifier being present in concentrations from 2 to 8% (m/m).

5. The composition of claim 1, wherein the W/O emulsifier is polyglyceryl-2-dipolyhydroxystearate in a concentration from 2 to 8% (m/m).

6. The composition of claim 1, wherein N-acyl amino acids, alkyl polyglycol ether citrates and/or alkyl and alkenyl oligoglycosides are present in a concentration from 0.1 to 10% (m/m) as anionic co-surfactants.

7. The composition of claim 1, wherein polyacrylates and/or polysaccharides are present in concentrations from 0.05 to 4% (m/m) as gelling agents.

8. The composition of claim 1, wherein optionally hydrotopic substances such as glycerol, ethanol, and/or propylene glycol are included.

9. The composition of claim 1, wherein optionally active ingredients of cosmetic dermatology are included.

10. The composition of claim 9, wherein the optional active ingredients of cosmetic dermatology are selected from the group of antioxidants, plant extracts, UV filters, urea, hyaluronic acid, provitamins and vitamins.

11. The composition of claim 1, wherein n-butane, isobutane, n-propane, n-pentane, isopentane, hexane, dimethyl ether, or mixtures thereof are used as foaming agents.

12. Aerosol cans, including a composition of claim 1.

13. A method for the preparation of a composition of claim 1, comprising the following steps:
weighing all lipids and optional lipophilic active ingredients and homogenizing in a first vessel;
adding the W/O emulsifier or the W/O emulsifiers under stirring;
presenting the glycerol in a second vessel and adding the gelling agent under vigorous stirring;
adding water and optional lipophilic active ingredients to the second vessel;
carefully adding co-surfactants while avoiding foam formation;
adding the lipophilic phase from the first vessel to the lipophilic phase in the second vessel, optionally adding water and finally homogenizing.

14. The method of claim 11, wherein all steps, namely
preparing the lipophilic phase,
preparing the hydrophilic phase,
adding the lipophilic phase to the hydrophilic phase,
are carried out at room temperature.
